(19) United States
(12) Patent Application Publication
(10) Pub. No.: US 2018/0160945 A1
(43) Pub. Date: Jun. 14, 2018

(54) POSTURE SENSING APPARATUS AND POSTURE SENSING METHOD

(71) Applicant: Industrial Technology Research Institute, Chutung (TW)

(72) Inventors: Shyh-Shyuan SHEU, Chutung (TW); Yu-Sheng CHEN, Chutung (TW); Chih-Sheng LIN, Chutung (TW); Li-Heng LEE, Chutung (TW); Chien-Yu WU, Chutung (TW)

(21) Appl. No.: 15/393,922

(22) Filed: Dec. 29, 2016

(30) Foreign Application Priority Data
Dec. 8, 2016 (TW) 105140633

(51) Int. Cl. A61B 5/11; G01D 5/14; G01D 5/16; A61B 5/00

(52) U.S. Cl. CPC A61B 5/1121; G01D 5/142; A61B 5/6802; A61B 5/1117; A61B 5/112; G01D 5/16

(57) ABSTRACT

A posture sensing apparatus and posture sensing method are provided. The posture sensing apparatus includes a magnetic object and a first magnetic sensor. The magnetic object and the first magnetic sensor are disposed respectively at two different positions of a user or a wearing object that the user is wearing. The magnetic object emits a magnetic field. The first magnetic sensor senses the magnetic field emitted by the magnetic object to generate a first angle, so as to obtain a posture of the user based on the first angle.

### CROSS-REFERENCE TO RELATED APPLICATIONS

This application claims priority to Taiwanese Patent Application No. 105140633, filed on Dec. 8, 2016. The entirety of the above-mentioned patent application is hereby incorporated by reference herein and made a part of this specification.

### BACKGROUND

### 1. Technical Field

The disclosure relates to posture sensing apparatuses and posture sensing methods.

### 2. Description of Related Art

There are several facts associated with aging. For example, the world's population is rapidly aging; there are 10% to 50% of a chance of falling among people who are older than 65 years old, and falling is one of the major causes for elderly accidental death; 8% of the elderly will get dementia, the chance of getting dementia increases by as much as twofold with every 5 years increase in age, and falls need to be prevented for people who suffer from dementia.

However, in the present industry, health care products are mostly centered on sports and fitness industry, such as wearable watches, bracelets and so on. These products are not developed from the perspective of the needs of the elderly or patients, and there are only a few health care products specifically focused on the problems and dangers that may be faced by the elderly and patients. Meanwhile, making these products more appealing to be worn by the elderly or patients is also needed. In addition, elderly or patient-related health care issues are important issues that medical staff and family members must face.

Therefore, there is a need for overcoming the aforementioned issues.

### SUMMARY

The disclosure provides a posture sensing apparatus and posture sensing method for sensing the posture of a user through magnetic object(s) and magnetic sensor(s).

The posture sensing apparatus according to the disclosure may include a magnetic object and a first magnetic sensor. The magnetic object is disposed on a user or a wearing object that the user is wearing, and the magnetic object emits a magnetic field. The first magnetic sensor is disposed on the user or the wearing object that the user is wearing, and the magnetic object and the first magnetic sensor are provided at different locations of the user or the wearing object. The first magnetic sensor senses the magnetic field emitted by the magnetic object to generate a first angle, so as to obtain the posture of the user based on the first angle.

The posture sensing method according to the disclosure may include: providing a magnetic object and a first magnetic sensor at different locations of a user or a wearing object that the user is wearing; emitting, by the magnetic object, a magnetic field; and sensing, by the first magnetic sensor, the magnetic field emitted by the magnetic object to generate a first angle, so as to obtain a posture of the user based on the first angle.

### BRIEF DESCRIPTION OF THE DRAWINGS

FIG. 1 is a graph illustrating a relationship between magnetic fields and distances;

FIG. 2 is a schematic diagram depicting a posture sensing apparatus arranged on a user or a wearing object that the user is wearing (not shown) according to the disclosure;

FIGS. 3A and 3B are schematic diagrams illustrating a posture sensing apparatus and a posture sensing method in accordance with a first embodiment of the disclosure;

FIG. 4 is a flowchart illustrating the posture sensing method in FIGS. 3A and 3B according to the disclosure;

FIGS. 5A and 5B are schematic diagrams illustrating a posture sensing apparatus and a posture sensing method in accordance with a second embodiment of the disclosure;

FIG. 6 is a schematic diagram illustrating another embodiment of the posture sensing apparatus; and

FIG. 7 is a flowchart illustrating the posture sensing method described in FIGS. 5A to 6 according to the disclosure.

### DETAILED DESCRIPTION OF THE EMBODIMENTS

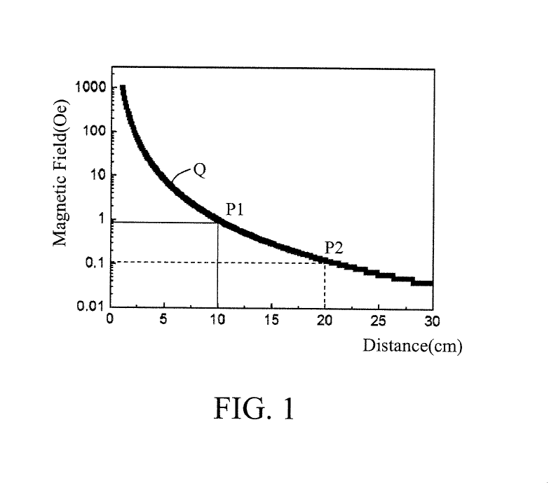

FIG. 1 is a graph illustrating a relationship between magnetic fields and distances. The magnetic field and the distance have a corresponding relationship indicated by a curve Q. For example, a distance of 10 cm at a location P1 on the curve Q corresponds to a magnetic field of about 1 Oe; and a distance of 20 cm at a location P2 on the curve Q corresponds to a magnetic field of about 0.1 Oe. Therefore, the disclosure exploits the corresponding relationship between the magnetic field and the distance of the curve Q to obtain a posture of a user.

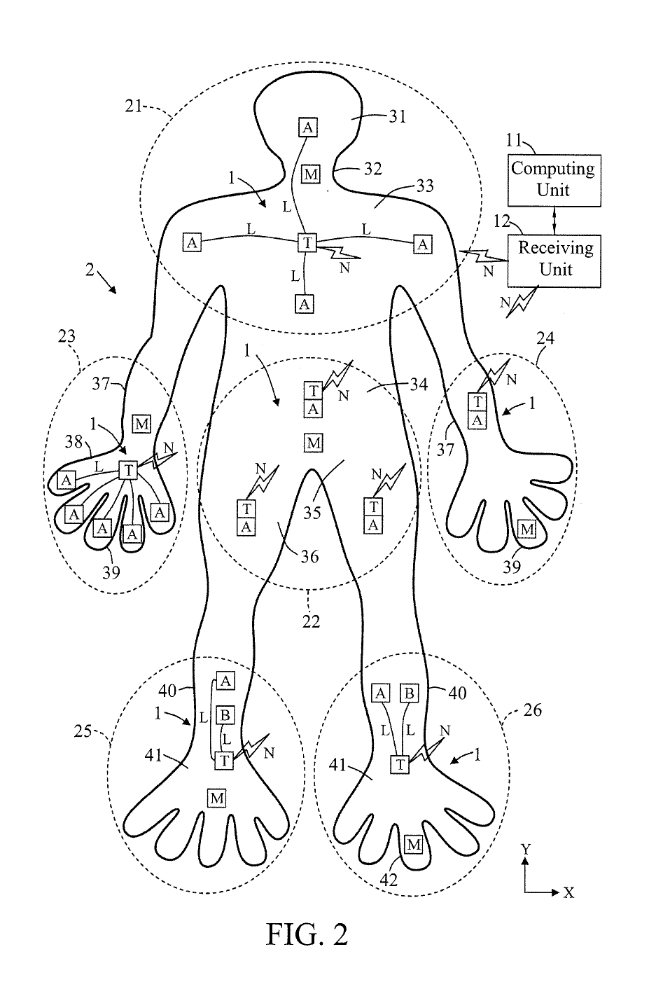

FIG. 2 is a schematic diagram depicting a posture sensing apparatus 1 arranged on a user 2 or a wearing object that the user is wearing (not shown) according to the disclosure. As shown, one or more (e.g., more than two) measurement areas can be allocated on the user 2, and one or more (e.g., two or more) posture sensing apparatuses 1 can be disposed respectively on the one or more measurement areas.

The user 2 can be a human or an animal. The wearing object that the user is wearing can be clothes, pants, a glove, a shoe, a sock, a hood, a necklace, a belt, a bracelet, a knee pad, a wrist pad, an ankle pad, a shoulder pads, a strap or any combination thereof. In an embodiment, there are six posture sensing apparatuses 1 and six corresponding measurement areas, including a first measurement area 21, a second measurement area 22, a third measurement area 23, a fourth measurement area 24, a fifth measurement area 25, and a sixth measurement area 26; however, the disclosure is not limited as such.

As an example, in the first measurement area 21, the posture sensing apparatus 1 may include a magnetic object M, four first magnetic sensors A, a transmitting unit T and four connecting wires L. The magnetic object M is disposed on the neck 32 (or a necklace) of the user 2. The four first magnetic sensors A are respectively disposed at four different locations of the head 31 (or a hood) and back 33 (or clothes) of the user 2. The transmitting unit T is disposed on the back 33 (or clothes) of the user 2, and is connected with the four first magnetic sensors A through the four connecting wires L.

In the second measurement area 22, the posture sensing apparatus 1 may include a magnetic object M, three first magnetic sensors A, and three transmitting units T. The magnetic object M is disposed on the buttock 35 (or pants) of the user 2. The three first magnetic sensors A and the three transmitting unit T are respectively combined into three modules, and these three modules are respectively disposed at three different locations of the waist 34 (or a belt) and thigh 36 (or pants) of the user 2.

In the third measurement area 23, the posture sensing apparatus 1 may include a magnetic object M, five first magnetic sensors A, a transmitting unit T, and five connecting wires L. The magnetic object M is disposed on an arm 37 (or clothes) of the user 2. The five first magnetic sensors A are respectively disposed at five different locations of fingers 39 (or a glove) of the user 2. The transmitting unit T is disposed on a palm 38 (or a glove) of the user 2, and is connected with the five first magnetic sensors A through the five connecting wires L.

In the fourth measurement area 24, the posture sensing apparatus 1 may include a magnetic object M, a first magnetic sensor A, and a transmitting unit T. The magnetic object M is disposed on a finger 39 (or a glove) of the user 2. The first magnetic sensor A and the transmitting unit T are combined into one module, and is disposed on an arm 37 (or clothes) of the user 2.

In the fifth measurement area 25, the posture sensing apparatus 1 may include a magnetic object M, a first magnetic sensor A, a second magnetic sensor B, a transmitting unit T and two connecting wires L. The magnetic object M is disposed on a foot 41 (or a sock) of the user 2. The first magnetic sensor A and the second magnetic sensor B are respectively disposed at two different locations of the same vertical plane (e.g., Y axis) on a calf 40 (or pants) of the user 2. The transmitting unit T is disposed on the foot 41 (or the sock) of the user 2, and is connected with the first magnetic sensor A and the second magnetic sensor B through the two connecting wires L, respectively.

In the sixth measurement area 26, the posture sensing apparatus 1 may include a magnetic object M, a first magnetic sensor A, a second magnetic sensor B, a transmitting unit T, and two connecting wires L. The magnetic object M is disposed on a toe 42 (or a sock) of the user 2. The first magnetic sensor A and the second magnetic sensor B are respectively disposed at two different locations of a calf 40 (or pants) of the user 2 on the same horizontal plane (e.g., X axis). The transmitting unit T is disposed on the foot 41 (or the sock) of the user 2, and is connected with the first magnetic sensor A and the second magnetic sensor B through the two connecting wires L, respectively.

In an embodiment, within the same measurement area of the user 2, only one magnetic object M is usually provided, thereby avoiding interference between the magnetic fields of the more than two magnetic objects M in the same measurement area. In another embodiment, only one transmitting unit T is provided on the user 2 or the wearing object that the user is wearing, and the transmitting unit T is connected with all of the first magnetic sensors A and the second magnetic sensors B through a plurality of connecting wires L.

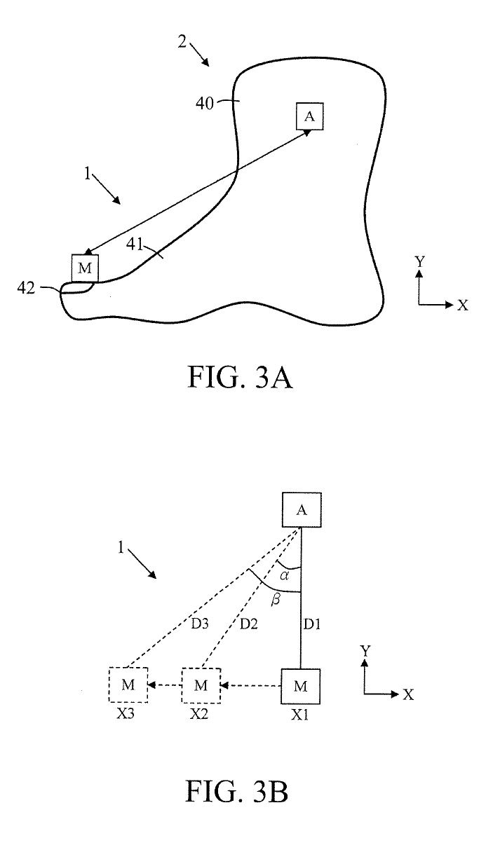

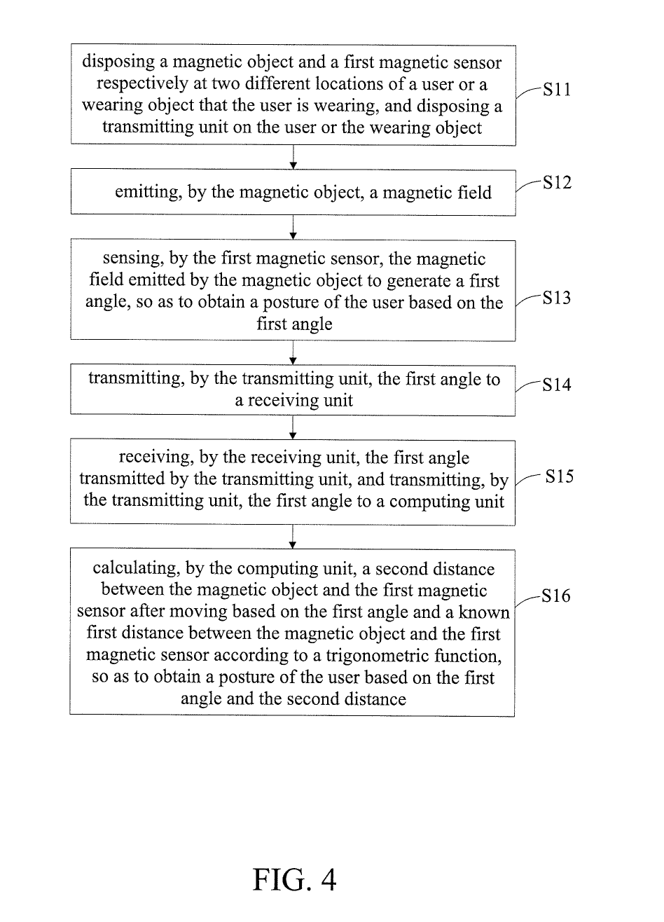

FIGS. 3A and 3B are schematic diagrams illustrating a posture sensing apparatus 1 and a posture sensing method in accordance with a first embodiment of the disclosure. FIG. 4 is a flowchart illustrating the posture sensing method in FIGS. 3A and 3B according to the disclosure. The following descriptions are to be understood in conjunction with FIGS. 1 and 2.

As shown in FIG. 3A, FIG. 3B and step S11 of FIG. 4, a magnetic object M and a first magnetic sensor A are disposed at different locations (or different measurement sites) of the user 2 or the wearing object that the user is wearing, and a transmitting unit T is disposed on the user 2 or the wearing object. In an embodiment, the magnetic object M is disposed on a toe 42 (or a sock) of the user 2, the first magnetic sensor A is disposed on a calf 40 (or pants) of the user 2, and the transmitting unit T is disposed on a foot 41 (or a sock) of the user 2 (see FIG. 2).

The magnetic object M can be a loadstone, a magnet, an electromagnet or the like. The first magnetic sensor A can be a Hall sensor, a tunneling magnetoresistance (TMR) sensor, a giant magnetoresistance (GMR) sensor, or an anisotropic magnetoresistance (AMR) sensor, and the Hall sensor can be a nine-axis sensor. The transmitting unit T can be a Bluetooth or WiFi-enabled transmitting unit; however, the disclosure is not limited to these.

As shown in FIG. 3A, FIG. 3B, and step S12 of FIG. 4, the magnetic object M emits a magnetic field.

As shown in FIG. 3A, FIG. 3B, and step S13 of FIG. 4, the first magnetic sensor A senses the magnetic field (or magnetic field intensity) emitted by the magnetic object M, and generates a first angle α based on the magnetic field (or magnetic field intensity), so as to obtain a posture of the measurement site (e.g., the toe 42) of the user 2 based on the first angle α.

Referring to FIG. 3B, assuming that the resolution of the first magnetic sensor A with respect to the magnetic field is 0.01 Oe, and the angle of the magnetic object M at location X1 is 0 degree. If the magnetic object M moves from location X1 (angle is 0 degree) towards location X2 along the X axis or the horizontal direction, the first magnetic sensor A senses that the change in magnetic field (i.e., the resolution) emitted by the magnetic object M is 0.01 Oe and converts it into an angle of 5 degrees. In other words, the first magnetic sensor A determines that the first angle α of the magnetic object M at location X2 is 5 degrees. As such, a posture of the toe 42 of the user 2 can be obtained.

As shown in FIG. 3A, FIG. 3B, and step S14 of FIG. 4, the transmitting unit T transmits the first angle α to a receiving unit 12 (see FIG. 2). The receiving unit 12 can be a signal receiver, a Bluetooth receiver, a wireless network receiver or antenna, or the like.

As shown in FIG. 3A, FIG. 3B, and step S15 of FIG. 4, the receiving unit 12 receives the first angle α transmitted by the transmitting unit T, and sends the first angle α to a computing unit 11 (see FIG. 2) through a wireless signal N. The computing unit 11 can be a processor (e.g., a CPU), an arithmetic logic unit (ALU), a computing software (e.g., an APP), or the like.

As shown in FIG. 3A, FIG. 3B, and step S16 of FIG. 4, based on the first angle α and a known first distance D1 between the magnetic object M (at location X1) and the first magnetic sensor A, the computing unit 11 calculates a second distance D2 between the magnetic object M (at location X2) and the first magnetic sensor A after moving according to a trigonometric function, so as to obtain a posture of the measurement site (e.g., the toe 42) of the user 2 based on the first angle α and the second distance D2.

Refer to FIG. 3B for example, the first magnetic sensor A determines that the first angle α is 5 degrees. It is assumed that the first distance D1 is known (e.g., 10 cm), and the computing unit 11 can then calculate the second distance D2 (e.g., 10.04 cm) using a trigonometric function (cos α=D1/D2), and obtains a posture of the toe 42 of the user 2 based on the first angle α (e.g., 5 degrees) and the second distance D2 (e.g., 10.04 cm).

In accordance with the steps S13 to S16 of FIG. 4, if the magnetic object M of FIG. 3B moves from location X2 (angle is 5 degrees) towards location X3 along the X-axis or the horizontal direction, the first magnetic sensor A senses that the change in magnetic field (i.e., the resolution) emitted by the magnetic object M is 0.01 Oe and converts it into an angle of 7 degrees. In other words, the first magnetic sensor A determines that a second angle 3 of the magnetic object M at location X3 is 7 degrees. As such, a posture of the toe 42 of the user 2 can be obtained. Furthermore, the first magnetic sensor A has determined that the second angle β is 7 degrees, assuming that the first distance D1 is known (e.g., 10 cm), and the computing unit 11 can then calculate a third distance D3 (e.g., 10.07 cm) using a trigonometric function (cos β=D1/D3), and obtains a posture of the toe 42 of the user 2 based on the second angle β (e.g., 7 degrees) and the third distance D3 (e.g., 10.07 cm).

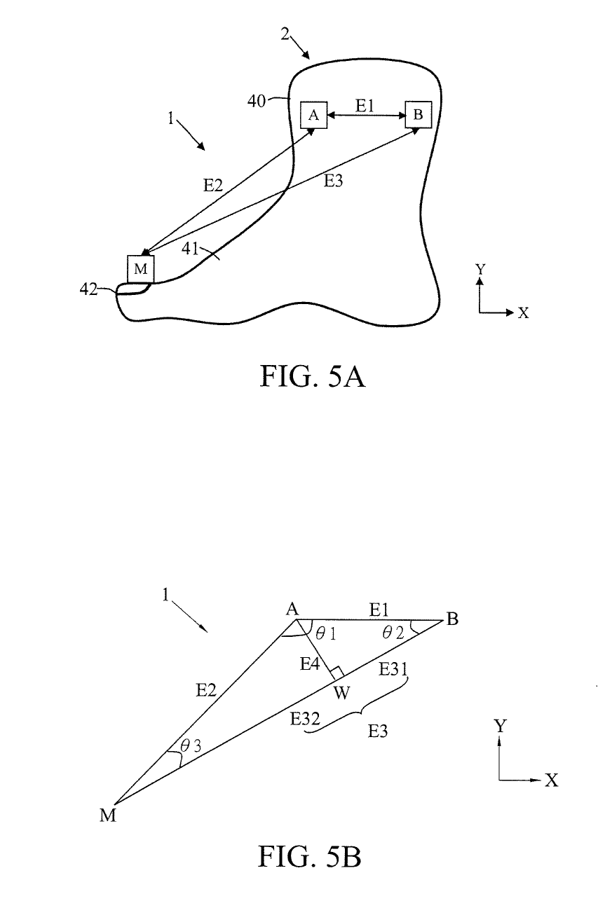

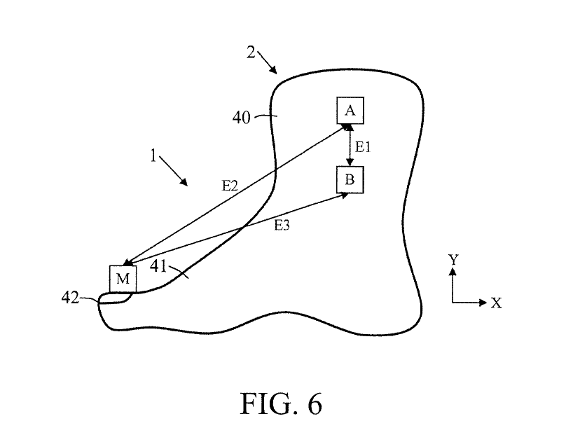

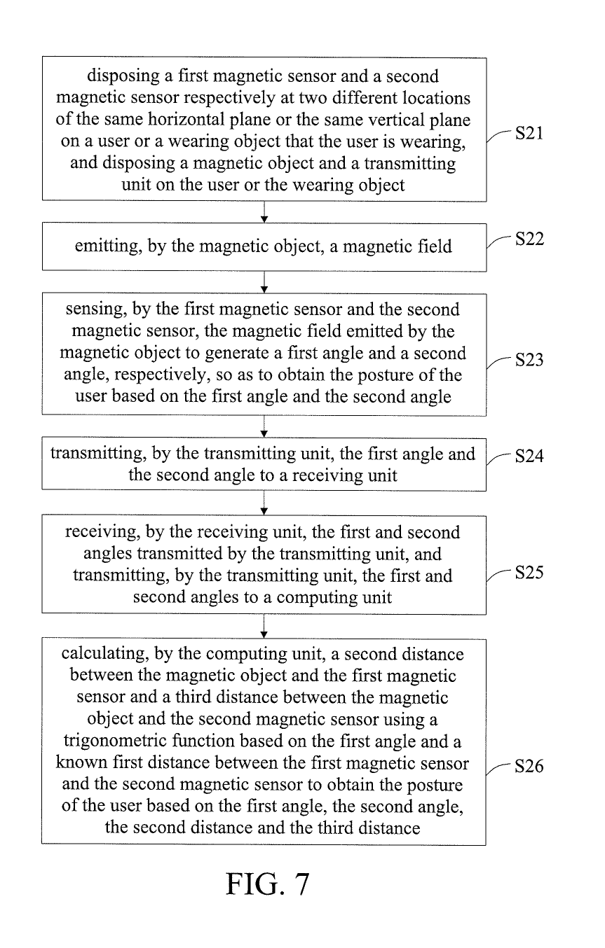

FIGS. 5A and 5B are schematic diagrams illustrating a posture sensing apparatus 1 and a posture sensing method in accordance with a second embodiment of the disclosure. FIG. 6 is a schematic diagram illustrating another embodiment of the posture sensing apparatus 1. FIG. 7 is a flowchart illustrating the posture sensing method described in FIGS. 5A to 6 according to the disclosure. The following descriptions are to be understood in conjunction with FIGS. 1 and 2.

As shown in FIG. 5A, FIG. 5B, FIG. 6, and step S21 of FIG. 7, a first magnetic sensor A and a second magnetic sensor B are respectively disposed on the same horizontal plane (e.g., the X-axis in FIG. 5A) or the same vertical plane (e.g., the Y-axis in FIG. 6) of the user 2 or the wearing object that the user is wearing, a magnetic object M and a transmitting unit T are disposed on the user 2 or the wearing object, and the first magnetic sensor A, the second magnetic sensor B, and the magnetic object M are provided at different locations (or different measurement sites) of the user 2 or the wearing object.

For example, the first magnetic sensor A and the second magnetic sensor B are respectively disposed at two different locations of the same horizontal plane (e.g., the X-axis in FIG. 5A) or the same vertical plane (e.g., the Y-axis in FIG. 6) of a calf 40 (or pants) of the user 2, the magnetic object M is disposed on a toe 42 (or a sock) of the user 2, and the transmitting unit T is disposed on a foot 41 (or a sock) of the user 2 (see FIG. 2).

As shown in FIG. 5A, FIG. 5B, FIG. 6, and step S22 of FIG. 7, the magnetic object M emits a magnetic field.

As shown in FIG. 5A, FIG. 5B, and step S23 of FIG. 7, the first magnetic sensor A and the second magnetic sensor B sense the magnetic fields emitted by the magnetic object M, and generate a first angle θ1 and a second angle θ2, respectively, based on the magnetic fields (or magnetic field intensity), so as to obtain a posture of the measurement site (e.g., the toe 42) of the user 2 based on the first angle θ1 and the second angle θ2.

Refer to FIG. 5B, for example, similar to the descriptions given with respect to FIG. 3B and step S13 of FIG. 4 above, the first magnetic sensor A and the second magnetic sensor B generate the first angle θ1 and the second angle 82, respectively, so as to obtain a posture of the toe 42 of the user 2 based on the first angle θ1 and the second angle θ2; further descriptions will not be repeated.

As shown in FIG. 5A, FIG. 5B, and step S24 of FIG. 7, the transmitting unit T transmits the first angle θ1 and the second angle 82 to a receiving unit 12 (see FIG. 2).

As shown in FIG. 5A, FIG. 5B, and step S25 of FIG. 7, the receiving unit 12 receives the first angle θ1 and the second angle 82 transmitted by the transmitting unit T, and sends the first angle θ1 and the second angle θ2 to a computing unit 11 (see FIG. 2) through a wireless signal N.

As shown in FIG. 5A, FIG. 5B, and step S26 of FIG. 7, based on the first angle θ1, the second angle θ2 and a known first distance E1 between the first magnetic sensor A and the second magnetic sensor B, the computing unit 11 calculates a second distance E2 between the magnetic object M and the first magnetic sensor A and a third distance E3 between the magnetic object M and the second magnetic sensor B according to a trigonometric function, so as to obtain a posture of the measurement site (e.g., the toe 42) of the user 2 based on the first angle θ1, the second angle θ2, the second distance E2 and the third distance E3.

Referring to FIG. 5B, the first magnetic sensor A and the second magnetic sensor B determine the first angle θ1 and the second angle θ2, respectively. Assuming that the first distance E1 is known, the computing unit 11 can then calculate a fourth distance E4 between the first magnetic sensor A and a location W using a trigonometric function (e.g., E4=E1*sin θ2), calculate a third angle θ3 that the magnetic object M resides by subtracting the first angle θ1 and the second angle θ2 from 180 degrees (i.e., θ3=180°−θ1−θ2), and further calculate the second distance E2 using a trigonometric function (e.g., sin θ3=E4/E2). Then, the computing unit 11 calculates a fifth distance E31 between the second magnetic sensor B and the location W using a trigonometric function (e.g., cos θ2=E31/E1), calculates a sixth distance E32 between the magnetic object M and the location W using a trigonometric function (e.g., cos θ3=E32/E2), and thus calculates the third distance E3 by adding the fifth distance E31 and the sixth distance E32 together (i.e., E3=E31+E32). Therefore, a posture of the toe 42 of the user 2 can be obtained based on the first angle θ1, the second angle θ2, the third angle θ3, the second distance E2, and the third distance E3.

Similarly, as described in FIG. 5B, the computing unit 11 may also calculate a second distance E2 and a third distance E3 in FIG. 6, and obtain a posture of the toe 42 of the user 2 based on the first angle θ1 (not shown), the second angle θ2, the third angle θ3, the second distance E2, and the third distance E3 of FIG. 6.

In the posture sensing apparatus and posture sensing method according to the disclosure, by employing magnetic object(s) in cooperation with magnetic sensor(s), a smaller or lighter posture sensing apparatus can be provided, the posture sensing apparatus can be arranged on a user or a wearing object that the user is wearing, and the magnetic field emitted by the magnetic object is sensed by the magnetic sensor(s) to generate information on angles and the distances, thereby obtaining the posture of measurement site(s) of the user (such as the head, the neck, the back, the hands, or the feet) for the purposes of physical rehabilitation, exercise training, posture correction, gait analysis, etc., to assist in the elderly care, patient treatment, illness prediction, sports training, or other applications.

It will be apparent to those skilled in the art that various modifications and variations can be made to the disclosed embodiments. It is intended that the specification and examples be considered as exemplary embodiments only, with a scope of the disclosure being indicated by the following claims and their equivalents.

What is claimed is:

1. A posture sensing apparatus, comprising:
a magnetic object configured for being disposed on a user or a wearing object that the user is wearing and emitting a magnetic field; and
a first magnetic sensor configured for being disposed on the user or the wearing object and sensing the magnetic field to generate a first angle, so as to obtain a posture of the user in accordance with the first angle,
wherein the magnetic object and the first magnetic sensor are provided at different locations of the user or the wearing object.

2. The posture sensing apparatus of claim 1, wherein the magnetic object is a loadstone, a magnet or an electromagnet.

3. The posture sensing apparatus of claim 1, wherein the user is a human or an animal, and the wearing object that the user is wearing is clothes, pants, a glove, a shoe, a sock, a hood, a necklace, a belt, a bracelet, a knee pad, a wrist pad, an ankle pad, a shoulder pads, a strap or any combination thereof.

4. The posture sensing apparatus of claim 1, further comprising a computing unit configured for calculating, based on the first angle and a known first distance between the magnetic object and the first magnetic sensor, a second distance between the magnetic object and the first magnetic sensor after the magnetic object has moved using a trigonometric function, so as to obtain the posture of the user based on the first angle and the second distance.

5. The posture sensing apparatus of claim 1, further comprising a second magnetic sensor, wherein the first magnetic sensor and the second magnetic sensor are respectively disposed at two different locations of the same horizontal plane or the same vertical plane on the user or the wearing object that the user is wearing, and the second magnetic sensor is configured for sensing the magnetic field emitted by the magnetic object to generate a second angle.

6. The posture sensing apparatus of claim 5, wherein at least one of the first magnetic sensor and the second magnetic sensor is a Hall sensor, a tunneling magnetoresistance (TMR) sensor, a giant magnetoresistance (GMR) sensor, or an anisotropic magnetoresistance (AMR) sensor.

7. The posture sensing apparatus of claim 5, further comprising a computing unit for calculating, based on the first angle and a known first distance between the first magnetic sensor and the second magnetic sensor, a second distance between the magnetic object and the first magnetic sensor and a third distance between the magnetic object and the second magnetic sensor using a trigonometric function, so as to obtain the posture of the user based on the first angle, the second angle, the second distance and the third distance.

8. The posture sensing apparatus of claim 1, further comprising a transmitting unit configured for being disposed on the user or the wearing object that the user is wearing and transmitting the first angle to a computing unit.

9. The posture sensing apparatus of claim 8, further comprising a receiving unit configured for receiving the first angle transmitted by the transmitting unit and transmitting the first angle to the computing unit.

10. A posture sensing method, comprising:
providing a magnetic object and a first magnetic sensor at different locations of a user or a wearing object that the user is wearing;
emitting, by the magnetic object, a magnetic field; and
sensing, by the first magnetic sensor, the magnetic field emitted by the magnetic object to generate a first angle, so as to obtain a posture of the user based on the first angle.

11. The posture sensing method of claim 10, wherein the magnetic object is a loadstone, a magnet or an electromagnet.

12. The posture sensing method of claim 10, wherein the user is a human or an animal, and the wearing object that the user is wearing is clothes, pants, a glove, a shoe, a sock, a hood, a necklace, a belt, a bracelet, a knee pad, a wrist pad, an ankle pad, a shoulder pads, a strap or any combination thereof.

13. The posture sensing method of claim 10, further comprising calculating, by a computing unit, based on the first angle and a known first distance between the magnetic object and the first magnetic sensor, a second distance between the magnetic object and the first magnetic sensor after the magnetic object has moved using a trigonometric function, so as to obtain the posture of the user based on the first angle and the second distance.

14. The posture sensing method of claim 10, further comprising disposing the first magnetic sensor and a second magnetic sensor respectively at two different locations of the same horizontal plane or the same vertical plane on the user or the wearing object that the user is wearing, and sensing, by the second magnetic sensor, the magnetic field emitted by the magnetic object to generate a second angle.

15. The posture sensing method of claim 14, wherein at least one of the first magnetic sensor and the second magnetic sensor is a Hall sensor, a tunneling magnetoresistance (TMR) sensor, a giant magnetoresistance (GMR) sensor, or an anisotropic magnetoresistance (AMR) sensor.

16. The posture sensing method of claim 14, further comprising calculating, by a computing unit, based on the first angle and a known first distance between the first magnetic sensor and the second magnetic sensor, a second distance between the magnetic object and the first magnetic sensor and a third distance between the magnetic object and the second magnetic sensor using a trigonometric function, so as to obtain the posture of the user based on the first angle, the second angle, the second distance and the third distance.

17. The posture sensing method of claim 10, further comprising disposing a transmitting unit on the user or the wearing object that the user is wearing, and transmitting, by the transmitting unit, the first angle to a computing unit.

18. The posture sensing method of claim 17, further comprising receiving, by a receiving unit, the first angle transmitted by the transmitting unit, and transmitting, by the transmitting unit, the first angle to the computing unit.
